(19) United States
(12) Patent Application Publication
(10) Pub. No.: US 2008/0045583 A1
(43) Pub. Date: Feb. 21, 2008

(54) STABLE LEVETIRACETAM COMPOSITIONS AND METHODS

(76) Inventors: David Delmarre, Vernon Hills, IL (US); Carlos-Julian Sison Centeno, Chicago, IL (US); Naga Mallika Surapaneni, Mundelein, IL (US); Danchen Gao, Chicago, IL (US)

Correspondence Address: WINSTON & STRAWN LLP;PATENT DEPARTMENT, 1700 K STREET, N.W., WASHINGTON, DC, 20006, US

(21) Appl. No.: 11/836,419

(22) Filed: Aug. 9, 2007

Related U.S. Application Data
(60) Provisional application No. 60/838,440, filed on Aug. 18, 2006.

(51) Int. Cl. A61K 31/40; A61P 25/08

(52) U.S. Cl. 514/424

(57) ABSTRACT

Stable, liquid compositions of levetiracetam that are substantially or entirely free of preservatives, particularly parabens, and/or sugars, such as natural sugars and sugar alcohols. The liquid compositions preferably include oral solutions or suspensions, and may include pharmaceutically acceptable excipients.

### CROSS REFERENCE TO RELATED APPLICATION

This application claims the benefit of co-pending U.S. provisional patent application No. 60/838,440, filed Aug. 18, 2006, the entire contents of which is hereby incorporated herein by express reference thereto.

### FIELD OF THE INVENTION

The invention relates to paraben- and sugar-free to pharmaceutical compositions of levetiracetam, a metabolite, or a pharmaceutically acceptable salt thereof, that are stable and of palatable flavor.

### BACKGROUND OF THE INVENTION

Levetiracetam is conventionally administered as an antiepileptic drug. The chemical name of levetiracetam, a single enantiomer, is (−)-(S)-α-ethyl-2-oxo-1-pyrrolidine acetamide, its molecular formula is C₈H₁₄N₂O₂ and its molecular weight is 170.21. Levetiracetam has the following structural formula:

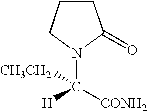

Levetiracetam is a white to off-white crystalline powder with a faint odor and a bitter taste. It is very soluble in water (104.0 g/100 mL). It is freely soluble in chloroform (65.3 g/100 mL) and in methanol (53.6 g/100 mL), soluble in ethanol (16.5 g/100 mL), sparingly soluble in acetonitrile (5.7 g/100 mL) and practically insoluble in n-hexane (solubility limits being expressed as g/100 mL solvent).

Keppra® brand levetiracetam is conventionally available as 250 mg, 500 mg and 750 mg tablets, and as a clear, colorless, grape-flavored liquid (100 mg/mL) for oral administration. Keppra® oral solution, for example, conventionally contains 100 mg of levetiracetam per mL with pharmaceutically inactive ingredients (i.e., excipients) including ammonium glycyrrhizinate, citric acid monohydrate, glycerin, maltitol solution, methylparaben, potassium acesulfame, propylparaben, purified water, sodium citrate dihydrate and natural and artificial flavor.

Conventional formulations of levetiracetam contain numerous preservatives and sugars that limit the applicability or desirability of the oral liquid dosage form of the drug. These conventional formulations include one or more parabens, such as methylparaben, butylparaben, ethylparaben, propylparaben and salts thereof. Parabens, however, have been linked to negative health effects, including drug allergies. See, Rowe, R. et al., The Handbook of Pharmaceutical Excipients, Pharmaceutical Press, Fourth Edition, 2003, pages 390-393 and 526-528. The paraben has been included to ensure the liquid dosage forms maintain stability of the composition.

The conventional formulations of levetiracetam also conventionally include one or more natural sugars. Sugar has typically been included, in addition to being a sweetener, to enhance the flavor of liquid compositions and to mask or minimize the bitter or unpleasant taste associated with certain drugs, excipients, or the combination thereof.

It is now desired to obtain stable liquid dosage forms of levetiracetam that avoid the problems of the prior art.

### SUMMARY OF THE INVENTION

The present invention can advantageously meet one or more of these unmet needs of the art by providing the inventive compositions and methods. The invention relates to liquid pharmaceutical compositions of levetiracetam having a pharmaceutically effective amount of levetiracetam, or a metabolite or a salt thereof, and a pharmaceutically acceptable carrier that is a liquid and which is substantially free, and preferably entirely free, of paraben, wherein the liquid pharmaceutical composition is stable. In a preferred embodiment, the invention relates to stable liquid pharmaceutical formulations that include a pharmaceutically effective amount of levetiracetam, or a metabolite or a salt thereof, and a pharmaceutically acceptable carrier that is a liquid and includes a preservative component present in an amount sufficient to provide stability to the formulation and which is substantially free of any paraben. In related embodiments, the composition is substantially free, and preferably entirely free, of preservatives that function only to preserve the levetiracetam.

The invention also relates to pharmaceutical compositions of levetiracetam in which the carrier is substantially free, and preferably entirely free, of sugar.

Yet another aspect of the invention relates to a liquid a solution adapted for oral administration. In some aspects, the pharmaceutically acceptable carrier includes excipients, such as a thickening agent, a non-sugar sweetening agent, a flavoring agent, a wetting agent, or a combination thereof. In at least some variations, the pharmaceutically acceptable carrier is aqueous and includes citric buffer, glycerin, propylene glycol, saccharin sodium, potassium acesulfame, and preferably a combination thereof. In a more preferred embodiment, the carrier also includes xylitol, sucralose, a flavoring agent, or a combination thereof. The flavoring agent may include artificial and/or natural flavor.

In a further aspect, the invention relates to methods of making a stable liquid pharmaceutical formulation in which a pharmaceutically acceptable carrier that is a liquid and is substantially, and preferably entirely, free of paraben is combined with a pharmaceutically effective amount of levetiracetam, or a metabolite or a salt thereof, with the carrier to provide a stable liquid formulation. In some variations, the carrier is also substantially, and preferably entirely, free of sugar.

In yet one other aspect, the invention relates to a method of administering a therapeutically effective amount of the substantially or entirely paraben free and/or sugar free levetiracetam composition of to prevent or treat epilepsy in a patient, preferably a human, and more preferably a pediatric human, in need of anti-epileptic treatment. In these variations, the paraben and sugar are substantially or entirely free.

Any of the embodiments illustrated herein stand independently, and any features or embodiments may be combined in any way, unless expressly excluded, to achieve a preferred embodiment. Additional advantages and embodiments of the invention will also become more apparent to those of ordinary skill in the art upon review of the teachings of the present application.

### DETAILED DESCRIPTION OF THE PREFERRED EMBODIMENTS

The present invention provides compositions of levetiracetam, methods of making thereof, and methods of administering thereof. More specifically, the present invention provides stable liquid compositions of levetiracetam improved compared to the art by being substantially, or preferably entirely, free of certain undesirable excipients, notably sugar, paraben(s), other preservative(s), or any combination thereof. As used herein, “sugar” means natural sugar, saccharide, and/or alcohol sugars, such as glucose, fructose, sucrose, tagatose, isomaltulose, lactitol, sorbitol, mannitol, trehalose, maltodextrin, polydextrose, erythritol, and maltol.

There has been increasing consumer demand for healthier pharmaceutical formulations and compositions. Moreover, many consumers prefer to avoid caloric intake, and consumers tend not to consider caloric content of medication but will be particularly eager to learn of formulations that avoid unnecessary calories while retaining a palatable flavor. It has now been discovered that stable liquid formulations of levetiracetam can be advantageously provided that are substantially or entirely free of, for example, methyl paraben, propylparaben, and/or sugar. The compositions are microbial resistant. For example, sugar-free formulations will be desirable for diabetics, pregnant women, and pediatric populations, three populations that typically are required or requested to limit sugar consumption. Moreover, the caloric content of sugar-free formulations of the invention is less than comparable sugar formulations.

In preferred embodiments, the compositions of the present invention are at least substantially free of these certain excipients, namely one or more parabens, preferably all parabens, sugar, other non-paraben preservatives, certain thickening agents, or any combination thereof. In a more preferred embodiment, the compositions of the present invention are at least substantially free of preservative excipients that provide only a preservative function. Thus, for example, methyl and propyl paraben each function only as a preservative, while glycerin and propylene glycol each have several additional functional benefits in addition to acting as a preservative. As used herein, “substantially free” is understood to be less than about 3 weight percent, preferably less than about 2 weight percent, and more preferably less than about 1 weight percent, of the undesired component. In a preferred embodiment, the term “substantially free” refers to less than about 0.5 weight percent, preferably less than about 0.15 weight percent, and more preferably less than about 0.05 or 0.01 weight percent. With respect to the excipient content, the amounts are with reference to the weight of the composition. As used herein, “completely free,” “entirely free,” or “free” is understood to mean an absence of, i.e., less than an analytically detectable amount, of the stated excipient.

A pharmaceutically effective amount of levetiracetam, which includes any metabolite or salt form thereof according to the invention, ranges from about 0.5% to about 30%, preferably from about 1% to about 20%, more preferably from about 5% to about 15%, and even more preferably from about 7.5 to about 12.5%, w/v of the pharmaceutical composition. An exemplary amount of levetiracetam includes about 10% w/v of the composition.

In one embodiment, the liquid carrier is substantially free of preservatives. As used in one variation of this embodiment, the term “preservative” in connection with pharmaceutical compositions of levetiracetam encompasses all conventional preservatives including, as would be known by one or ordinary skill in the art, including parabens, with the exception of propylene glycol, polyethylene glycol (“PEG”), and glycerin; preferably the term preservative, as used herein, includes even PEG.

As used in another variation of this embodiment, the term “preservative” encompasses all preservatives that only, or preferably primarily, function to preserve the composition, such as to provide antimicrobial activity. Examples of a preservatives functioning only to preserve the composition are sodium benzoate, butylated hydroxy toluene, butylated hydroxyanisole, ethylenediamine tetraacetic acid, paraoxybenzoic acid esters, methyl, ethyl, butyl, and propyl parabens, chlorobutanol, benzyl alcohol, phenylethylalcohol, dehydroacetic acid, sorbic acid, benzalkonium chloride (BKC), benzethonium chloride, phenol, phenylmercuric nitrate, thimerosal, and any combination thereof. In accordance with the present invention, under this definition, excipients such as parabens are included, while excipients such as propylene glycol, which may function as a solvent, for instance, and glycerin, which may function as a sweetening agent, for instance, are not included. Thus, in this embodiment, all components that act as their sole function as a preservative are preferably excluded from the formulations of the invention.

Preferred embodiments of the present invention include amounts of propylene glycol, glycerin, or a combination, in the liquid carrier. Preferably, both are included. In some embodiments, the formulation may include glycerin but no glycol if the glycerin concentration is greater than about 20%. In other embodiments, the formulation may include propylene glycol but no glycerin if the propylene glycol concentration is greater than about 15%. Preferred formulations of the invention include at least 20% glycerin, at least 15% propylene glycol, or both. Without being bound by theory, it is believed that including sufficient amounts of certain components, such as glycerin and glycol can provide a preservative effect to the composition while at the same time providing other functionality including stabilizing the levetiracetam.

Propylene glycol when included is typically present in an amount from about 5% to 90%, preferably from about 10% to 80%, more preferably from about 15% to 60% w/v of the composition. In a most preferred embodiment, the propylene glycol is present in an amount of about 17.5% to about 40% w/v of the composition. In an exemplary embodiment, the propylene glycol includes about 20% w/v of the pharmaceutical composition. Glycerin, when included, is preferably present in an amount from about 10% to 50%, preferably from about 20% to 40%, and more preferably from about 25% to 35%, v/v of the pharmaceutical composition. In an exemplary embodiment, glycerin is present at about 27% to 33%, preferably about 30% v/v of the composition.

The present invention is characterized by the stability of the liquid carriers that are substantially or entirely free of a paraben, preferably all parabens, or even all preservatives. In one embodiment, the stability is greater than conventional levetiracetam formulations and compositions, particularly any oral solutions, that contain paraben. In another embodiment, the liquid compositions degrade at most about 20% faster than conventional paraben-containing formulations, more preferably at most about 10% faster, and most preferably are substantially equivalent in stability (or even more stable as noted above). Stability is measured in the formulations of the present invention by storing samples under predetermined temperature, e.g., 40° C. to 50° C. and humidity conditions, e.g., at a relatively low humidity, such as below about 30%, or at ambient humidity conditions for 4 to 12 weeks. Standard procedures such as HPLC or UV spectroscopic methods are used to determine the amount of active ingredient remaining before and after storage to compare degradation and stability.

Preferably, the present formulation is sufficiently stable to be stored under ambient temperature conditions, e.g., at room temperature of 20° C. or 72° F., as well as under cooler conditions including refrigeration. The stability preferably provides a shelf life of at least 120 days, preferably 180 days, and more preferably at least one year even at room temperature.

In a preferred embodiment, the formulations of the invention surprisingly and unexpectedly provide an antimicrobial effect despite being at least substantially free, more preferably entirely free, of parabens, and preferably despite being substantially free, more preferably entirely free, of all preservatives, in the formulation. “Anti-microbial effectiveness” or “microbial resistant” as used herein refers to the inhibition, management, or delayed growth of bacteria or other microbes in the formulation over time.

The final dosage form most preferably retains assay limits of about 90% to 110% of the original assay value when stored under controlled room temperature conditions. Some embodiments of the present invention exhibit stability of at least about 95%, preferably about 98%, more preferably at least about 99%, even more preferably at least about 99.5%, and most preferably at least about 99.9%, at 8 weeks at either 40° C. or 50° C. at low or ambient relative humidity at 8 and 12 weeks.

In preferred embodiments, at 5 weeks, at 40° C., the degradation of the levetiracetam composition is less than about 0.2%, preferably less than about 0.1%, more preferably less than about 0.05%, even more preferably less than about 0.025%, and in a most preferred embodiment less than or equal to about 0.01%. In other preferred embodiments, at 5 weeks, at 50° C., the degradation of the levetiracetam composition is less than about 0.2%, preferably less than about 0.15%, and more preferably less than equal to about 0.12%.

Preferably, the liquid composition of the present invention is adapted for oral administration and is in the form of an oral solution or suspension, preferably aqueous based. Most preferably, the liquid form is an aqueous solution. In some embodiments, the oral solution contains from about 80 to 120 mg of levetiracetam per mL. In a preferred embodiment, the oral solution contains about 100 mg of levetiracetam per mL.

The pharmaceutically acceptable carrier can include an amount of one or more conventional pharmaceutically inert excipients in an amount and of a type that do not substantially detrimentally affect stability or lack of sugar in the inventive formulations. Such excipients may include, but are not limited to, a thickening agent, a non-sugar sweetening agent, a flavoring agent, taste-masking component, a wetting agent, and a coloring agent. It should be understood by those of ordinary skill in the art that excipients may be substituted, added, and replaced in the compositions of the present invention so as to comply with the preservative-free, paraben-free, and/or sugar free aspects of the invention. Thus, if a conventional excipient has a significant preservative effect, it would not be considered to be a part of a substantially preservative-free embodiment of the invention.

Suitable concentrations of sweetening agent can be selected by those of skill in the art based on published information, manufacturers' data sheets, etc., and by routine testing depending on what is included therein. In a typical embodiment, the sweetening agent includes one or more sugars and/or saccharide sugars that do not require preservatives to inhibit degradation of the sugar in the formulation, i.e., artificial sugars or xylitol. In a preferred embodiment, the sweetening agent may include one or more artificial sweetening agents and/or xylitol. In a most preferred embodiment, the only sweetening agent included in the formulation of the present invention is one or more artificial sweeteners. For example, the sweetening agent may include one of more of: acesulfame and salts thereof, e.g., acesulfame potassium, alitame, aspartame, neotame, cyclamate, saccharin and salts thereof, e.g., saccharin sodium, neohesperidin dihydrochalcone, stevioside, thaumatin, etc., and sucralose, and any combination thereof.

Preferably, in some variations, the sweetening agent includes a saccharin, and more preferably includes sodium saccharin. Preferably, in some variations, the sweetening agent includes acesulfame, and more preferably includes acesulfame potassium. In certain preferred embodiments, the sweetening agent including an acesulfame-containing component, sucralose-containing component, a saccharin-containing component, or combination thereof. Preferably, all three are included in the sweetening agent. It should be understood that, in the substantially or entirely sugar-free embodiments of the invention, the composition is preferably at least substantially free or entirely free of all saccharide and alcohol sugars, preferably all natural sugars with the exception of xylitol (which may be included).

The sweetening agent is preferably present by itself, or in combination with a taste-masking component, in an amount sufficient to mask any off-flavors in the taste of the drug, or salt or metabolite thereof, and preferably to also mask any other off-flavor components included in the formulation if desired. The sweetening agent is typically present in an amount from about 0.01% to 5%, preferably from about 0.2% to 4%, and more preferably from about 0.4% to 0.6%, w/v of the composition formulation. In a most preferred embodiment, the sweetening agent is present in an amount of about 0.45% to 0.55 w/v of the composition formulation.

A suitable taste-masking component or artificial sweetness enhancer agent may be added to the composition as necessary. One exemplary taste-masking agent is Sweet-Am® brand by Flavors of North America. This excipient may be present in an amount from about 0.01% to 2%, preferably from about 0.1% to 1%, and more preferably from about 0.2% to about 0.4%, v/v of the composition.

One or more thickening agents are typically included in liquid pharmaceutical formulations, such as to provide a better mouthfeel or texture or even to help with dispersion or stability. Without being bound by theory, however, it is believed that the presence of one or more thickening agents in a formulation typically requires the addition of one or more parabens or other preservatives to the formulation. Accordingly, in one embodiment, the formulation of the present invention is substantially free, and preferably entirely free, of a thickening agent. In one preferred embodiment, the liquid formulation is substantially or entirely free of a thickening agent in addition to minimizing the presence of any significant amounts of paraben, preservative, or sugar, or any combination.

A thickening agent or viscosity-enhancing agent that may be used without the need for a preservative may be optionally included to generally improve the mouth-feel of the composition, the formulation stability, and/or to help coat the lining of the gastrointestinal tract to facilitate administration. Thickening agents that require the use of preservatives to maintain stability and/or antimicrobial effect include, for example, various cellulose based compounds, gelatin, starches, and gums. Thickening compounds to be particularly minimized in the formulation include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxymethyl cellulose, hydroxypropyl cellulose, hydroxypropyl methyl cellulose (“HPMC”), starch, sodium starch glycolate, starch tragacanth, and xanthan gum, or a combination thereof. Preferably, the composition is at least substantially, more preferably entirely, free of any of these above-noted thickening agents. Preferred thickening agents, when used, include polyvinyl alcohol, povidone, propylene carbonate, or a combination thereof, without necessarily requiring that a substantial amount of a preservative component be present. A thickening agent may, however, be included in liquid formulations of the invention. These formulations do not need to include a thickening agent, but may if desirable.

A suitable buffering agent, as understood in the art, is used to adjust the pH of the liquid carrier. An exemplary buffering agent includes one or more of potassium phosphate dibasic, calcium carbonate, sodium bicarbonate, sodium and potassium hydroxide, or any combination thereof. In embodiments of the present invention, the buffer is present in an amount from about 25% to 75%, preferably from about 35% to 65%, and more preferably at about 45% to 55% v/v of the composition.

Citric buffer is included in the preferred buffering agent. In preferred embodiments, the citric buffer includes sodium citrate dihydrate, citric acid, or a combination thereof. The pH range in the solution is about 4.5 to 6.5, and preferably about 5.5 to 6. The preferred pH is about 5.6 to 5.8.

Any suitable, sugar-free flavoring agent available to those of ordinary skill in the art may be included in the present liquid carrier, typically to enhance patient compliance by making the compositions of the present invention more palatable. The flavoring agent is one or more substances capable of enhancing taste or aroma of a composition. Any suitable natural or synthetic flavoring agent can be selected, such as those listed in standard reference books, for example Fenaroli's Handbook of Flavor Ingredients, 3rd edition (1995). The flavoring agent is typically selected in type and amount to increase palatability, e.g., by decreasing or eliminating any undesired taste or off-flavors in the taste, i.e., a taste mask, that would otherwise be detectable by the patient to whom the compositions are administered. Examples of a suitable flavoring agent, when used, include one or more of menthol, peppermint, anise, and any fruit flavor, such as one or more of grapefruit, orange, grape, lemon, lime, mango, strawberry, pineapple, or cherry, or a combination thereof. Such a flavoring agent, if present, may be present at a concentration in the composition of about 0.1 to 5 mg/ml, preferably about 0.2 to 3 mg/ml, and most preferably about 0.5 to 2 mg/ml. In certain embodiments, the flavor agent is present in amount from about 0.1% to 0.5%, and more preferably at about 0.3%, v/v of the pharmaceutical composition.

A colorant agent, when included, may be provided in an amount sufficient to provide the compositions with a more aesthetic and/or distinctive appearance. Any suitable colorant agent available to those of ordinary skill in the art may be selected. Typically, a colorant agent suitable for inclusion in the present invention includes one or more synthetic organic food additives (e.g., food dyes such as food red dye Nos. 2 and 3, food yellow dye Nos. 4 and 5 and food blue dye Nos. 1 and 2), water-insoluble lake dyes (e.g., aluminum salts of the above synthetic organic food additives, etc.), and natural pigments (e.g., beta-carotene, chlorophyll, iron oxide red, etc.), or any combination thereof. Suitable colorants include D&C Red No. 33, FD&C Red No. 3, FD&C Red No. 40, D&C Yellow No. 10, and C Yellow No. 6. Any combination of these or any other suitable colorant agent can be included. Such a colorant agent, if present, may be present in a conventional amount.

A wetting agent, or surfactant, may be included in the liquid compositions of the present invention. When used, the wetting agent may include any suitable type available to those of ordinary skill in the art. For example, the wetting agent may include one or more quaternary ammonium compounds, such as benzalkonium chloride, benzethonium chloride and cetylpyridinium chloride; TPGS (i.e., d-alpha tocopheryl polyethylene glycol 1000 succinate), dioctyl sodium sulfosuccinate; polyoxyethylene alkylphenyl ethers, such as nonoxynol 9, nonoxynol 10, and octoxynol 9; poloxamers (polyoxyethylene and polyoxypropylene block copolymers); polyoxyethylene fatty acid glycerides and oils, such as polyoxyethylene (8) caprylic/capric mono- and diglycerides (e.g., Labrasol®, Gattefosse), polyoxyethylene (35) castor oil and polyoxyethylene (40) hydrogenated castor oil; polyoxyethylene alkyl ethers, such as polyoxyethylene (20) cetostearyl ether; polyoxyethylene fatty acid esters, such as polyoxyethylene (40) stearate; polyoxyethylene sorbitan esters, such as polysorbate 20 and polysorbate 80 (e.g., Tween® 80, ICI); propylene glycol fatty acid esters, such as propylene glycol laureate (e.g., Lauroglycol®, Gattefosse); sodium lauryl sulfate; fatty acids and salts thereof, such as oleic acid, sodium oleate and triethanolamine oleate; glyceryl fatty acid esters, for example glyceryl monostearate; sorbitan esters, such as sorbitan monolaurate, sorbitan monooleate, sorbitan monopalmitate and sorbitan monostearate; tyloxapol; lecithin; stearyl triethanolamine; laurylaminopropionic acid; and any combination thereof. Such a wetting agent, if present, may be used in a conventional amount, based on the total volume of the pharmaceutically acceptable carrier.

In embodiments of the present invention, the levetiracetam compositions of the present invention are preferably administered in conventional amounts to treat, manage, and/or prevent epilepsy or epileptic episodes as would be understood by one of ordinary skill in the art. The composition is administered to humans, including adult and pediatric populations. The compositions of the present invention are administered between one and four times daily, preferably between two and three times daily, and most preferably two times daily. The doses administered typically range from about 250 mg to 4000 mg daily, preferably from about 500 mg to 3500 mg daily, and more preferably from about 1000 mg to 3000 mg daily.

In preferred embodiments, compositions of levetiracetam are initially administered to human patients are initiated with a daily dose equivalent to 1000 mg/day, given as twice-daily dosing (i.e., 500 mg BID). Additional dosing increments may be given 1000 mg/day additional every 2 weeks) to a daily dose of 3000 mg.

In pediatric patients, i.e., between about 4 to 16 years of age, preferred administrations initially include a daily dose of 20 mg/kg in 2 divided doses (i.e., 10 mg/kg BID). In some variations, the daily dose is increased every 2 weeks by increments of about 20 mg/kg to a daily dose of about 60 mg/kg (i.e., 30 mg/kg BID). If a patient cannot tolerate a daily dose of about 60 mg/kg, the daily dose may be reduced. In the clinical trial, the mean daily dose was 52 mg/kg. Patients with body weight ≦20 kg should be dosed with oral liquid formulation of the invention.

The following calculation can be used by those of ordinary skill in the art to determine the appropriate daily dose of oral solution for pediatric patients, based on a daily dose of 20 mg/kg/day, 40 mg/kg/day or 60 mg/kg/day:

Total daily dose (mL/day)=[Daily dose (mg/kg/day)×patient weight (kg)]/100 mg/mL

The bioavailability of levetiracetam is not affected by food. Accordingly, in preferred embodiments, the levetiracetam composition of the present invention is administered orally, either with or without food.

An exemplary formulation of the present invention includes: about 1% to 20% w/v levetiracetam, or a pharmaceutically acceptable metabolite or salt thereof; about 20% to 80% v/v of citric buffer that preferably includes sodium citrate dihydrate, citric acid, or a combination thereof; from about 20% to 50% v/v of glycerin; from about 15% to 90% w/v of propylene glycol; a flavoring agent present in amount from about 0.01% to 5% v/v; and from about 0.3% to 7.5% w/v of a sweetening agent formed from one or more artificial sugars, xylitol, or a combination thereof. Preferably, this formulation is at least substantially free, more preferably it does not include any analytically detectable amount, of paraben, preservative, natural sugar (not including xylitol), or a combination thereof. Preferably, the sweetening agent includes about 0.1 to 2% w/v of saccharin sodium, about 0.1% to 0.5% w/v of potassium acesulfame, 0.1 to 5% w/v of sucralose, or more preferably a combination of two or more of these sweetening agents at least substantially free of natural sugar. In a more preferred embodiment the composition above contains no detectable natural sugar or preservative.

The term “about,” as used herein, should generally be understood to refer to both numbers in a range of numerals. Moreover, all numerical ranges herein should be understood to include each whole integer within the range.

### EXAMPLES

The invention is further defined by reference to the following illustrative examples that may be used to prepare or administer the compositions of the present invention. These examples are for illustrative purposes only, and are not to be construed as limiting the appended claims.

### Example 1

### Formulation without Flavor According to the Invention

An exemplary formulation is as follows:

Citric buffer (pH=5.7): 50% (v/v)

Glycerin: 30% (v/v)

Propylene Glycol: 20% (v/v)

Levetiracetam: 10% (w/v)

Saccharin Sodium: 0.5% (w/v)

Acesulfame Potassium: 0.5% (w/v)

Sweet-Am® flavor/sweetener: 0.2% (w/v)

(commercially available from Flavors of North America)

### Example 2

### Formulation with Flavor According to the Invention

A flavor is added to the formulation as in Example 1. The citric buffer is reduced and included in an amount of 49.7% v/v of the composition, with 0.3% v/v of grape flavor added.

### Example 3

### Stability Data According to the Invention

The samples were analyzed by HPLC.

TABLE 1
Summarized Stability Data

| | | | Percent |
|---|---|---|---|
| Formulation | Temperature (° C.) | Time (Days) | Degradation |
| Formulation as in | 50 | 38 | 0.12 |
| Example 1 | | | |
| Formulation as in | 40 | 38 | 0.01 |
| Example 1 | | | |

This surprising and unexpectedly demonstrated that, despite substantially or entirely eliminating paraben and sugar from the formulation, stability was achieved even under accelerated testing conditions.

### Example 4

### Stability Data According to the Invention

The anti-microbial effectiveness of the composition based on Example 1 was tested over time. The formulation was inoculated with a microorganism and incubated at 20-25° C. for 28 days. The protocol was based on based on USP<51>. Table 2 presents data for 14 and 28 days for five bacterial strains. A table with antimicrobial effectiveness should be included based on the formulation described in example 1.

TABLE 2
Anti-Microbial Effectiveness Data

| Inoculation | No. of Log Reductions | No. of Log Reductions |
|---|---|---|
| Microorganism | After 14 days | After 28 days |
| E. coli | 4.1 | 4.1 |
| E. aeruginosa | 4.4 | 4.4 |
| S. aureus | 4.3 | 4.3 |
| C. albicans | 4.1 | 4.1 |
| A. niger | 1.5 | 1.5 |

Although preferred embodiments of the invention have been described in the foregoing description and figures, it will be understood that the invention is not limited to the specific embodiments disclosed herein but is capable of numerous modifications by one of ordinary skill in the art. It will be understood that the materials used and the chemical and pharmaceutical details may be slightly different or modified from the descriptions herein without departing from the methods and compositions disclosed and taught by the present invention.

What is claimed:

1. A stable liquid pharmaceutical formulation that comprises:
a pharmaceutically effective amount of levetiracetam, or a metabolite or a salt thereof; and
a pharmaceutically acceptable carrier that is a liquid and comprises a preservative component present in an amount sufficient to provide stability to the formulation and which is substantially free of any paraben.

2. The pharmaceutical composition of claim 1, wherein the pharmaceutically acceptable carrier is at least substantially free of any saccharide sugar.

3. The pharmaceutical composition of claim 2, wherein the pharmaceutically acceptable carrier is entirely free of any saccharide sugar.

4. The pharmaceutical composition of claim 2, wherein the pharmaceutically acceptable carrier is at least substantially free of paraben.

5. The pharmaceutical composition of claim 1, wherein the pharmaceutically acceptable carrier is entirely free of paraben.

6. The pharmaceutical composition of claim 2, wherein the composition is a solution adapted for oral administration.

7. The pharmaceutical composition of claim 2, wherein the pharmaceutically acceptable carrier comprises a non-sugar sweetening agent, a flavoring agent, taste-masking component, a wetting agent, or a combination thereof.

8. The pharmaceutical composition of claim 7, wherein the flavoring agent comprises a natural and/or artificial flavor.

9. The pharmaceutical composition of claim 7, wherein the flavoring agent is present in amount from about 0.01% to 5% v/v.

10. The pharmaceutical composition of claim 1, wherein the preservative component comprises glycerin, propylene glycol, or a combination thereof.

11. The pharmaceutical composition of claim 7, wherein the pharmaceutically acceptable carrier comprises citric buffer, glycerin, propylene glycol, saccharin sodium, potassium acesulfame, sucralose, water, or combinations thereof.

12. The pharmaceutical composition of claim 7, which comprises:
from about 1% w/v to 20% w/v of levetiracetam;
about 20% to 80% v/v of citric buffer;
from about 10% to 50% v/v of glycerin;
from about 5% to 90% w/v of propylene glycol;
from about 0.1% to 2% w/v of saccharin sodium; and
from about 0.1% to 5% w/v of potassium acesulfame.

13. The pharmaceutical composition of claim 2, wherein the levetiracetam is present in an amount from about 1% to 20% w/v.

14. The pharmaceutical composition of claim 13, wherein the levetiracetam is present in an amount from about 7.5% to 12.5% w/v.

15. The pharmaceutical composition of claim 11, wherein the citric buffer is present in an amount sufficient to buffer the solution to a pH level of about 4.5 to 6.5.

16. The pharmaceutical composition of claim 15, wherein the citric buffer comprises sodium citrate dihydrate, citric acid, or a combination thereof.

17. The composition of claim 2, wherein the degradation of levetiracetam is less than about 0.2%, as measured by HPLC after 38 days at 50° C.

18. The composition of claim 2, wherein the degradation of levetiracetam is less than about 0.1%, as measured by HPLC after 38 days at 40° C.

19. The composition of claim 1, wherein the pharmaceutically acceptable carrier comprises a thickening agent that is polyvinyl alcohol, povidone, propylene carbonate, propylene glycol alginate, sodium alginate, or a combination thereof.

20. The composition of claim 1, wherein the composition is at least substantially free of a thickening agent.

21. A method of preparing a stable liquid pharmaceutical formulation that comprises:
providing a pharmaceutically acceptable carrier that is a liquid, comprises a sufficient amount of a preservative component to impart stability to the formulation, and is substantially free of paraben; and
combining a pharmaceutically effective amount of levetiracetam, or a metabolite or a salt thereof, with the carrier to provide the stable liquid formulation.

22. The method of claim 21, wherein the carrier is at least substantially free of any preservative component that provides only a preservative effect.

23. A method of administering a therapeutically effective amount of the composition of claim 2 to prevent or treat epilepsy in a patient in need of anti-epileptic treatment.

24. The method of administering the composition of claim 23, wherein the patient is a human.

25. The method of administering the composition of claim 24, wherein the patient is a pediatric human.

26. The method of administering the composition of claim 23, wherein the composition is administered one to four times daily.
